SEQUENCE LISTING (4 sequences)

SEQ ID NO: 1 - DNA, 1746 bp, Artificial Sequence
  synthetic
atggttcaatctcactatgactttattatcgttggtggtggcacggctggtaacacggttgcgggtcgcctggcagaaaatccggacgtctcgattctggtggttgaagcgggcgtggccaacagctctgaactgccggaaatcaccacgccgagcaacgcgatgaatctgcgtagttccaaacatgattgggcctacaaaaccacgctggtgaaacgtgatgactatgaacgcattgaaaaaccgaacacccgcggcaaagcactgggcggttcatcgagcctgaattactttacctggatcccgggttgcaaaccgacgttcgatcgttgggctgaatatggcggtaaagaatggacgtgggacccgctggttccgtatctgcgcaaatctgtcacctaccatgatgacgccggcctgtataatccggaactgaagaaagtgggcggcggcggcccgattccgatctctcacagtgaactggtgaacgaactgaaaccgtttcgtgataatctgattaaagcatggaaatctaccggtaaaccgctgacggaaaacatttacgatggcgaaatgatcggtctgaatcattgtattagtaccatctaccacggcaaacgctccggttcatacctgtttctgcagaacaccccgaatgtcacgattatcccggaagtgcattccaaaaccctgattatcgatgcatcacacacggctaaaggcgtcgtggttattgacaaagaaggtaatgaacattccttttatgcgacccgtgaagtgatcgtttctcaaggcgtgttcgaaagtccgaaactgctgatgctgagcggcattggtccgcgtaaagaactggaatcgaacggtatcgaagtcaaagtggaaagccgccatgttggccagaatctgctggatcacccgggtgttccgtttgtcctgcaagtgaaagacggtgtgtgcgcagatgacattctgatgcgtccgaccgcgcagaacaaagcggccatcgcccagtaccaaaaagatggttcgggtgcagtgggtagcggtctgctggaaattgttggctttccgcgcatcgatgaatacttcgaaaaagacccgctgtatcgtgaacgcaaagcagctaatggcggtaaagatccgttttgtccggagggtcagccgcattttgaactggatttcgttggcatgtatggtaccgcgtttcaatggcatttcccgaccccgaagaaaggcagccacattaccgtcgtggttgatctggttcgtccggtctctgacccgggtgaagtgaccctgaatagtgcagatccgctggaagaaccgaaaattaacctgaactttttcgctgatgacctggatattgttggcatgcgtgaaggtatccgctttagctatgatgtcctgaccaaaggcacgggttttaaagacctggtcgtgaaagaattcccgtgggaaatgccgctggatgacgaaaaagaaatgaaacgtgccgtgctggatcgctgccagaccgcattccacccgtgtggtacggctcgtctgagtaaaaacattgatcaaggcgttgtcgacccggcgctgaaagtgcatggtgttaaacacctgcgtgttatcgatgcctccattatcccgattatcccggactgccgcattcagaattcagtgtatatgatcggtgaaaaaggcgcagacctgattaaagcagaccacaaagacctgtataaacatcaccaccaccaccat

SEQ ID NO: 2 - protein, 582 aa, Artificial Sequence
  synthetic
MVQSHYDFIIVGGGTAGNTVAGRLAENPDVSILVVEAGVANSSELPEITTPSNAMNLRSSKHDWAYKTTLVKRDDYERIEKPNTRGKALGGSSSLNYFTWIPGCKPTFDRWAEYGGKEWTWDPLVPYLRKSVTYHDDAGLYNPELKKVGGGGPIPISHSELVNELKPFRDNLIKAWKSTGKPLTENIYDGEMIGLNHCISTIYHGKRSGSYLFLQNTPNVTIIPEVHSKTLIIDASHTAKGVVVIDKEGNEHSFYATREVIVSQGVFESPKLLMLSGIGPRKELESNGIEVKVESRHVGQNLLDHPGVPFVLQVKDGVCADDILMRPTAQNKAAIAQYQKDGSGAVGSGLLEIVGFPRIDEYFEKDPLYRERKAANGGKDPFCPEGQPHFELDFVGMYGTAFQWHFPTPKKGSHITVVVDLVRPVSDPGEVTLNSADPLEEPKINLNFFADDLDIVGMREGIRFSYDVLTKGTGFKDLVVKEFPWEMPLDDEKEMKRAVLDRCQTAFHPCGTARLSKNIDQGVVDPALKVHGVKHLRVIDASIIPIIPDCRIQNSVYMIGEKGADLIKADHKDLYKHHHHHH

SEQ ID NO: 3 - DNA, 1745 bp, Artificial Sequence
  synthetic
catatggctaccgatggatctcacttcgacttcgttattgtgggcggcggcacggcgggtaacaccgtggccggccgtcttgctgaaaaccccaatgtcacggtcttgatcgtcgaggcgggtatcgggaaccctgaggatatcccagagattactacaccttcctctgctatggaccttcgtaacagcaagtacgactgggcctataaaaccaccatggttaggcgcgatgactacgaacgtatcgagaagccgaacacacgtggaaagacccttggtggcagttcatcactcaattacttcacatgggttccaggccataaggctacctttgaccaatgggaagaatttggcggcaaggagtggacttgggatccccttgttccttaccttcgcaagagtgctacctatcacgatgaccccaggctttactcgcctgagcttgagaagatcggtggtggtggccctattccaatctctcatgctgagttgatcgatgagatggcgcccttccgtgagaacctcaccaaggcttggaagtccatgggccagcctttgattgaaaacatctatgatggtgaaatggatggtcttacccactgctgtgataccatctaccgaggccagcggtctggcagtttcctgtttgtcaagaataagcccaacatcacaattgtgcccgaggttcattctaagcgccttattattaacgaggccgaccgcacttgcaagggtgttactgttgtgaccgccgctggcaacgagttgaatttcttcgcggaccgtgaagttatcctttcccaaggtgtcttcgagacccctaagcttcttatgctcagcggtatcggacccacccgtgagctctcgagacatggtatcaacaccatcgtcgactctcgccacgtcggccagaacttgatggatcacccaggtgttcccttcgtgctgcgcgtcaaagacggcttcggtatggacgacgttcttctgcgccacggccctaagagagatgccgtcgtttccgcctataacaaaaatcgctccggccctgttggctccggtctactcgaactggtcggattcccccgtatcgacaagtatctcgagaaggatgccgaataccgcaaggcgaaagctgccaacggaggcaaggacccattttcgcccttgggacaacctcacttcgagctcgacttcgtctgcatgttcggtaccgccttccaatggcacttccccacccccaagaccggtgatcaccttaccgtggtcgttgacctggtccgccctatctccgaccctggtgaggtgaccttgaacagtgccgacccctttcagcagccgaatatcaaccttaatttcttcgccaatgacctcgatatcattgccatgcgtgagggtatcagattcagctatgacttgctgttcaagggcgagggcttcaaggacctggttgagagcgagtacccatgggaaatgcctctcgactccgacaaggagatgcacagagccgttctggaccgttgccagactgccttccacccgactggtactgcccgtctctcgaagaatatcgatcagggtgtggtggacccgaagctcaaggtccacggtatcaagaagctccgtgttgctgatgcttcggttattcctatcatccctgactgccgtatccagaactctgtctacgctgtcggtgagaagtgtgccgatatgatcaaggccgagcacaaggatctttacgcggccgc

SEQ ID NO: 4 - protein, 589 aa, Artificial Sequence
  synthetic
MATDGSHFDFVIVGGGTAGNTVAGRLAENPNVTVLIVEAGIGNPEDIPEITTPSSAMDLRNSKYDWAYKTTMVRRDDYERIEKPNTRGKTLGGSSSLNYFTWVPGHKATFDQWEEFGGKEWTWDPLVPYLRKSATYHDDPRLYSPELEKIGGGGPIPISHAELIDEMAPFRENLTKAWKSMGQPLIENIYDGEMDGLTHCCDTIYRGQRSGSFLFVKNKPNITIVPEVHSKRLIINEADRTCKGVTVVTAAGNELNFFADREVILSQGVFETPKLLMLSGIGPTRELSRHGINTIVDSRHVGQNLMDHPGVPFVLRVKDGFGMDDVLLRHGPKRDAVVSAYNKNRSGPVGSGLLELVGFPRIDKYLEKDAEYRKAKAANGGKDPFSPLGQPHFELDFVCMFGTAFQWHFPTPKTGDHLTVVVDLVRPISDPGEVTLNSADPFQQPNINLNFFANDLDIIAMREGIRFSYDLLFKGEGFKDLVESEYPWEMPLDSDKEMHRAVLDRCQTAFHPTGTARLSKNIDQGVVDPKLKVHGIKKLRVADASVIPIIPDCRIQNSVYAVGEKCADMIKAEHKDLYAAALEHHHHHH

(19) United States
(12) Patent Application Publication
(10) Pub. No.: US 2018/0321202 A1
(43) Pub. Date: Nov. 8, 2018

(54) METHODS AND DEVICES FOR DETECTING METHANOL POISONING USING FORMATE OXIDASE

(71) Applicant: OSLO UNIVERSITETSSYKEHUS HF, Oslo (NO)

(72) Inventors: Knut Erik HOVDA, Oslo (NO); Gaut GADEHOLT, Oslo (NO); Dag JACOBSEN, Oslo (NO)

(21) Appl. No.: 15/773,743

(22) PCT Filed: Nov. 4, 2016
(86) PCT No.: PCT/IB2016/001734

§ 371 Date: May 4, 2018

Related U.S. Application Data
(60) Provisional application No. 62/252,006, filed on Nov. 6, 2015.

(51) Int. Cl. G01N 31/00; G01N 33/573; C12Q 1/28; G01N 33/72; G01N 33/52

(52) U.S. Cl. CPC G01N 31/005; G01N 33/573; C12Q 1/28; C12Y 111/01007; G01N 33/52; G01N 2333/90203; G01N 33/725

(57) ABSTRACT

The present disclosure relates to compositions and methods for diagnosis, research, and screening for chemicals in biological fluids (e.g., related to methanol poisoning). In particular, the present disclosure relates to point of care systems and methods for detecting formic acid or formate, in biological fluids by means of natural or recombinant formate oxidase.

### FIELD OF THE INVENTION

The present disclosure relates to compositions and methods for diagnosis, research, and screening for chemicals in biological fluids related to methanol poisoning. In particular, the present disclosure relates to using an enzyme with formate oxidase activity in point of care systems for detecting formic acid or formate in biological fluids.

### BACKGROUND OF THE INVENTION

Methanol poisoning affects thousands each year. Fifteen to fifty percent of the exposed persons die, and a sizeable proportion of the remaining victims are left with permanent blindness, impaired vision or brain injury. In an outbreak in the Czech Republic in 2012 more than 120 people were poisoned, and 40 people died. In an outbreak in Kenya in May 2014 more than 350 people were poisoned, and more than 100 people died.

The patients typically present with metabolic acidosis, which is an unspecific condition with a number of different possible causations. Methanol poisoning is a treatable condition. Specific treatment is necessary. But because the condition is infrequent, it is commonly overlooked because specific diagnosis can only be carried out in a few laboratories capable of measuring methanol, formic acid/formate, or both. In these laboratories establishing the analysis and maintaining the competence incur considerable investment and running costs.

What is needed are rapid and accurate analytical methods that do not require heavy investment in instruments or competence. A system for bedside analysis, at the point of care, will give an early diagnosis and will trigger specific treatment at the earliest possible time.

### SUMMARY OF THE INVENTION

The present disclosure relates to compositions and methods for diagnosis, research, and screening for chemicals in biological fluids related to methanol poisoning. In particular, the present disclosure relates to using an enzyme with formate oxidase activity in point of care systems for detecting formic acid or formate in biological fluids.

In some embodiments, the present invention provides an assay device for measuring formate/formic acid, comprising: a test strip comprising a) an oxidase enzyme (formate oxidase) which produces hydrogen peroxide while oxidizing the formate with atmospheric oxygen; b) a peroxidase, for example horseradish peroxidase, capable of oxidizing suitable substrates with the generated hydrogen peroxide; and c) a suitable substrate for the peroxidase, serving as a precursor of an indicator dye to be read photometrically by reflex photometry or fluorimetrically in a reading instrument specially constructed for the purpose, or visually. The present invention is not limited to a particular indicator dye. In other embodiments the peroxidase substrate is a substance which after oxidation forms a product with a redox potential distinctively different from the bacground. On yet other embodiments the generated hydrogen peroxide is read directly, facilitated by judicious choice of sensor electrode functionalities providing one or more redox mediators.

The present invention is not limited to a particular material for construction of the test strip. Examples include, but are not limited to, nitrocellulose membranes, nylon membranes, or mixed polymer membrane CQ (IPOC). In some embodiments, the test strip further comprises a sample application pad. In some embodiments, the test strip further comprises a carbohydrate (e.g., trehalose, sucrose and/or dextran). In some embodiments, the test strip further comprises a surfactant (e.g., BioTerge AS 40). In some embodiments, the test strip further comprises bovine serum albumin. In some embodiments, the test trip in encased in a housing (e.g., plastic housing) comprising at least one viewing window.

Additional embodiments provide a kit, comprising any of the aforementioned assay devices. In some embodiments, the kit comprises a first test strip comprising formate oxidase, peroxidase and indicator substrate.

Further embodiments provide the use of any of the aforementioned kits to detect a toxin or a metabolite thereof (e.g., formic acid or formate) in a biological sample. Embodiments of the present invention provide a system, comprising: any of the aforementioned kits; and an apparatus or device for detection of hydrogen peroxide (e.g., flow through assay).

In further embodiments, the present invention provides a method for detecting the methanol metabolite, formate, in a biological sample from a subject, comprising: a) contacting a biological sample with a formate oxidase enzyme that oxidizes the formate into carbon dioxide, and with hydrogen peroxide as byproduct that is quantifiable by the secondary reagent system consisting of a peroxidase and an indicator precursor substrate. In some embodiments, the biological sample is blood (e.g., whole blood), serum, plasma, or urine. In some embodiments, the oxidase enzyme and the secondary system are embedded in a test strip (e.g., constructed of a synthetic material). In some embodiments, the final indicator dyes are detected spectrophotometrically by a portable dedicated instrument, or visually, or by means of a laboratory-based stationary or semi-mobile system.

In some embodiments, the presence of formic acid in the biological sample is indicative of methanol poisoning in the subject. In some embodiments, the method further comprises the diagnostic step necessary to justify the cost, effort and possible risk of treating the subject for methanol poisoning when formic acid is present in the biological sample. In some embodiments, the treatment is ethanol or fomepizole. In some embodiments, ethanol is administered at a rate intended to provide a concentration in the blood of the patient of 70-130 mg/dL. In some embodiments, the method is completed in three hours or less (e.g., two hours or less, one hour or less, 30 minutes or less, 15 minutes or less, or 5 minutes or less).

In some embodiments, the present invention provides a method for detecting formic acid in a biological sample from a subject, comprising: a) contacting a biological sample with formate oxidase and generating hydrogen peroxide, with the subsequent reaction of hydrogen peroxide with the indicator dye precursor by means of the catalytic influence of a peroxidase.

In further embodiments, the present invention provides for use of formate oxidase to diagnose or detect methanol poisoning in a subject, wherein the formate oxidase acts on formate in a biological sample to produce hydrogen peroxide and the hydrogen peroxide is detected by producing a colored reagent in the presence of a peroxidase enzyme and an indicator dye precursor and the production of the colored reagent is indicative of methanol poisoning in the subject. In some embodiments, the formate oxidase is selected from the group consisting of an Aspergillus formate oxidase, a Debaromyces formate oxidase, and a Paecillomyces formate oxidase. In some embodiments, the formate oxidase is a recombinant formate oxidase. In some embodiments, the formate oxidase exhibits activity upon reconstitution from a dried form on a solid or porous substrate. In some embodiments, the biological sample is blood, serum, plasma, or urine. In some embodiments, the indicator dye precursor is selected from TMB and ABTS. In some embodiments, the peroxidase enzyme is horse radish peroxidase. In some embodiments, the formate oxidase, the peroxidase and the indicator dye precursor are embedded in a test strip. In some embodiments, the test strip is selected from the group consisting of nitrocellulose membranes, nylon membranes, and mixed polymer membrane CQ (IPOC). In some embodiments, the test strip forms a flow through assay. In some embodiments, the colored reagent is detected photometrically. In some embodiments, the colored reagent is detected using a blood glucose meter or blood cholesterol meter. In some embodiments, the colored reagent is detected visually. In some embodiments, the formate oxidase is used to detect formate in a concentration in the biological sample of from 0.1 mM to 20 mM, 0.5 mM to 20 mM, and 1.0 mM to 20 mM, and preferably from 0.1 mM to 12 mM, 0.5 mM to 12 mM, and 1.0 mM to 12 mM. In some embodiments, a detectable amount of the colored reagent is produced within from about 30 seconds to 10 minutes. In some embodiments, the amount of colored reagent produced is quantitative for the amount of formate in the biological sample.

In some embodiments, the present invention provides methods for detecting methanol poisoning in a subject suspected of being poisoned with methanol, comprising: a) contacting a biological sample from the subject with a formate oxidase enzyme wherein the formate oxidase acts on formate in the biological sample to produce hydrogen peroxide; and b) detecting the hydrogen peroxide by producing a colored reagent by reacting the hydrogen peroxide with a peroxidase enzyme and an indicator dye precursor to produce the colored reagent; wherein the production of the colored reagent is indicative of methanol poisoning in the subject. In some embodiments, the formate oxidase is selected from the group consisting of an Aspergillus formate oxidase, a Debaromyces formate oxidase, and a Paecillomyces formate oxidase. In some embodiments, the formate oxidase is a recombinant formate oxidase. In some embodiments, the formate oxidase exhibits activity upon reconstitution from a dried form on a solid or porous substrate. In some embodiments, the biological sample is blood, serum, plasma, or urine. In some embodiments, the indicator dye precursor is selected from TMB and ABTS. In some embodiments, the peroxidase enzyme is horse radish peroxidase. In some embodiments, the formate oxidase, the peroxidase and the indicator dye precursor are embedded in a test strip. In some embodiments, the test strip is selected from the group consisting of nitrocellulose membranes, nylon membranes, and mixed polymer membrane CQ (IPOC). In some embodiments, the test strip forms a flow through assay. In some embodiments, the colored reagent is detected photometrically. In some embodiments, the colored reagent is detected using a blood glucose meter or blood cholesterol meter. In some embodiments, the colored reagent is detected visually. In some embodiments, the formate oxidase is used to detect formate in a concentration in the biological sample of from 0.1 mM to 20 mM, 0.5 mM to 20 mM, and 1.0 mM to 20 mM, and preferably from 0.1 mM to 12 mM, 0.5 mM to 12 mM, and 1.0 mM to 12 mM. In some embodiments, a detectable amount of the colored reagent is produced within from about 30 seconds to 10 minutes. In some embodiments, the amount of colored reagent produced is quantitative for the amount of formate in the biological sample. In some embodiments, the methods further comprise the step of treating the subject for methanol poisoning when formic acid is present in the biological sample. In some embodiments, the treatment is ethanol or fomepizole.

In some embodiments, the present invention provides methods for detecting formate in a biological sample from a subject, comprising: a) contacting a biological sample with formate oxidase such that formate in the biological sample reacts with the formate oxidase to generate hydrogen peroxide; and b) detecting the hydrogen peroxide.

In some embodiments, the present invention provides a device comprising a substrate having thereon dried formate oxidase in an amount sufficient to oxidize formate in a biological sample when contacted with the biological sample in the presence of a reporting system to produce a detectable signal corresponding to the presence of formate in the sample. In some embodiments, the reporting system comprises a peroxidase enzyme and an indicator dye precursor. In some embodiments, the peroxidase enzyme and the indicator dye precursor are dried onto the substrate. In some embodiments, the formate oxidase is selected from the group consisting of an Aspergillus formate oxidase, a Debaromyces formate oxidase, and a Paecillomyces formate oxidase. In some embodiments, the formate oxidase is a recombinant formate oxidase. In some embodiments, the substrate is porous substrate. In some embodiments, the porous substrate is a test strip. In some embodiments, the test strip is selected from the group consisting of nitrocellulose membranes, nylon membranes, and mixed polymer membrane CQ (IPOC). In some embodiments, the porous substrate comprises a sample receptive surface. In some embodiments, the porous substrate further comprises a carbohydrate. In some embodiments, the carbohydrate is trehalose and/or dextran. In some embodiments, the porous substrate further comprises a surfactant. In some embodiments, the surfactant is BioTerge AS 40. In some embodiments, the porous substrate further comprises bovine serum albumin. In some embodiments, the formate oxidase, the peroxidase and the indicator dye precursor are embedded in the test strip. In some embodiments, the test strip forms a flow through assay. In some embodiments, the biological sample is blood, serum, plasma, or urine. In some embodiments, the indicator dye precursor is selected from the group consisting of TMB (3,3′,5,5′-tetramethylbenzidine), ABTS (2,2′-Azinobis [3-ethylbenzothiazoline-6-sulfonic acid]-diammonium salt) and a dye or detection reagent with a peroxidase substrate group capable of yielding an indicator dye. In some embodiments, the peroxidase enzyme is horse radish peroxidase. In some embodiments, the formate oxidase is provided in an amount sufficient to detect formate in a concentration in the biological sample of from 0.1 mM to 20 mM, 0.5 mM to 20 mM, and 1.0 mM to 20 mM, and preferably from 0.1 mM to 12 mM, 0.5 mM to 12 mM, and 1.0 mM to 12 mM. In some embodiments, the formate oxidase is provided in an amount sufficient to cause the production of detectable amount of the colored reagent within from about 30 seconds to 10 minutes.

In some embodiments, the present invention provides an assay device, comprising: a porous substrate comprising a) a formate oxidase polypeptide; b) a peroxidase enzyme; and c) an indicator dye precursor.

In some embodiments, the present invention provides for use of the described assay devices for detection of methanol poisoning (via the formate intermediate) in a subject.

In some embodiments, the present invention provides a kit comprising an assay device as described herein. In some embodiments, the kit comprises a first test strip comprising formate oxidase. In some embodiments, the kit further comprises a container with a format standard solution. In some embodiments, the present invention provides for use of the kit to detect the presence of formate or methanol poisoning in a subject.

Additional embodiments of the present disclosure are provided in the description and examples below.

### DESCRIPTION OF THE FIGURES

FIG. 1 shows detection of formate using formate oxidase.

FIG. 2 shows nucleic acid and amino acid sequences of formate oxidase from Debaryomyces vanrijae; SEQ ID NO:1 (DNA sequence), SEQ ID NO:2 (protein sequence).

FIG. 3 shows testing of formate concentrations.

FIG. 4 shows testing of formate concentrations.

FIG. 5 Shows nucleic acid and amino acid sequences of formate oxidase from Aspergillus oryzae, Genbank ID XM_001727326.1 (SEQ ID NO:3 (DNA sequence), SEQ ID NO:4 (protein sequence).

### DEFINITIONS

Unless defined otherwise, all terms of art, notations and other scientific terms or terminology used herein have the same meaning as is commonly understood by one of ordinary skill in the art to which this disclosure belongs. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

As used herein, “a” or “an” means “at least one” or “one or more.”

As used herein, the terms “detect”, “detecting” or “detection” may describe either the general act of discovering or discerning or the specific observation of a detectable composition.

The term “dry reagent test strip” refers to an analytical device in the form of a test strip, in which a test sample fluid, suspected of containing an analyte, is applied to the strip (which is frequently made of porous materials such as paper, nitrocellulose, and cellulose). The test fluid and any suspended analyte can flow along or through the strip to a reaction zone in which the analyte (if present) interacts with a detection agent or detection system to indicate a presence, absence and/or quantity of the analyte.

The term “sample application area” refers to an area where a fluid sample is introduced to a test strip, such as a dry reagent test strip described herein or other assay device. In one example, the sample may be introduced to the sample application area by external application, as with a dropper or other applicator. In another example, the sample application area may be directly immersed in the sample, such as when a test strip is dipped into a container holding a sample. In yet another example, the sample may be poured or expressed onto the sample application area.

The term “solid support” or “substrate” means material which is insoluble, or can be made insoluble by a subsequent reaction. Numerous and varied solid supports are known to those in the art and include, without limitation, nitrocellulose, the walls of wells of a reaction tray, multi-well plates, test tubes, polystyrene beads, magnetic beads, membranes, microparticles (such as latex particles) and red blood cells. Any suitable porous material with sufficient porosity to allow access by reagents and a suitable surface affinity to immobilize reagents and/or analyte is contemplated by this term. For example, the porous structure of nitrocellulose has excellent absorption and adsorption qualities for a wide variety of reagents. Nylon possesses similar characteristics and is also suitable. Microporous structures are useful, as are materials with gel structure in the hydrated state.

Further examples of useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood (e.g., whole blood), blood products, such as plasma, serum, urine, saliva, sputum, and the like. Such examples are not however to be construed as limiting the sample types applicable to the present invention.

### DETAILED DESCRIPTION OF THE INVENTION

The present disclosure relates to compositions and methods for diagnosis, research, and screening for chemicals (e.g., toxins or metabolites thereof) in biological fluids (e.g., related to methanol poisoning). In particular, the present disclosure relates to point of care systems and methods for detecting formic acid or formate, and other clinically relevant chemicals in biological fluids.

Formic acid/formate is the toxic (poisonous) metabolite of methanol, and without the formation of this methanol would not be toxic to humans (d′Alessandro et al., Env. Health Perspectives 102:168 1994; Hovda et al., J. Analytical Toxicology 29 2005; each of which is herein incorporated by reference in its entirety). Treatment of methanol poisoning utilizes inhibitors of the metabolism of methanol to formic acid.

Very few options for detecting methanol poisoning are available. Methanol analyses are expensive and not easily accessible (e.g., only a few centers in Norway are performing them; in New York, analysis takes several days; and in the developing world, it often takes several weeks if it at all is possible). Alternative indirect methods exist (e.g., osmolality measurements), but they are nonspecific, and almost never available outside the Western world.

Embodiments of the present disclosure provide solutions for the lack of rapid (e.g., less than several hours and preferably less than several minutes), cost effective testing for methanol poisoning in the field at the point of care. In some embodiments, the present invention provides simplified methods for detecting clinically relevant chemicals in biological fluids (e.g., formic acid or formate) that utilize a formate oxidase enzyme.

In some embodiments, the present invention provides systems and methods for detection of formic acid or formate to detect methanol poisoning. The systems and methods described herein are simple, inexpensive, rapid, and utilize existing hardware.

### I. Assay Devices, Kits, and Systems

In some embodiments, the present invention provides assays and assay devices for the detection and diagnosis of methanol poisoning in a subject. In some preferred embodiments, the assays and assay devices are able to detect the level of formate (or formic acid) in a biological sample (e.g., saliva, blood or plasma). Formate or formic acid is the toxic agent produced by metabolism of methanol by mammals including humans.

In some preferred embodiments, the assays and assay devices of the present invention utilize a formate oxidase (FOX) enzyme. Formate oxidase enzymes are NAD independent enzymes that catalyze the oxidation of formate into carbon dioxide and hydrogen peroxide as shown in the following reaction:

HCOO⁻(formate)+O₂+H⁺→CO₂+H₂O₂

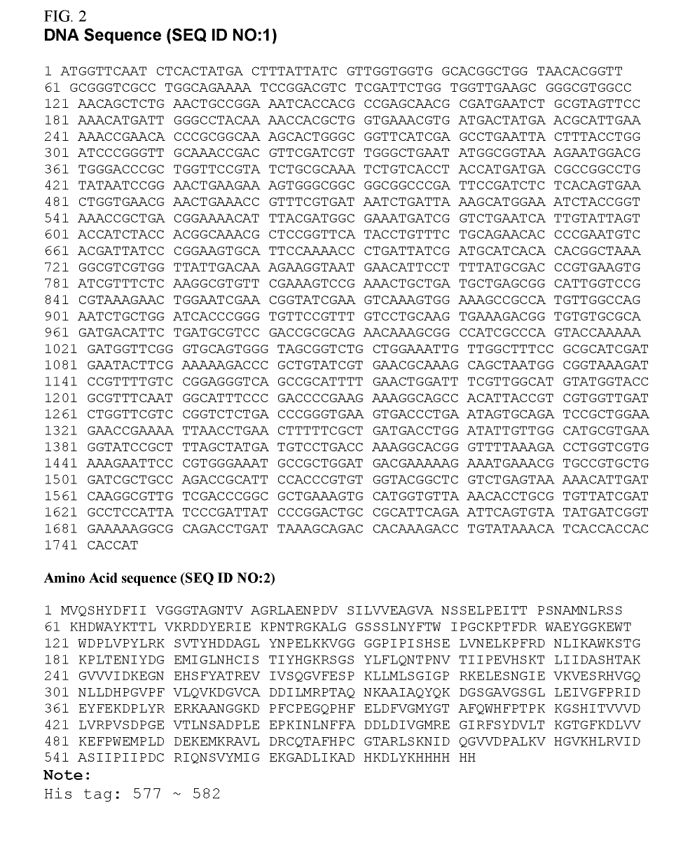

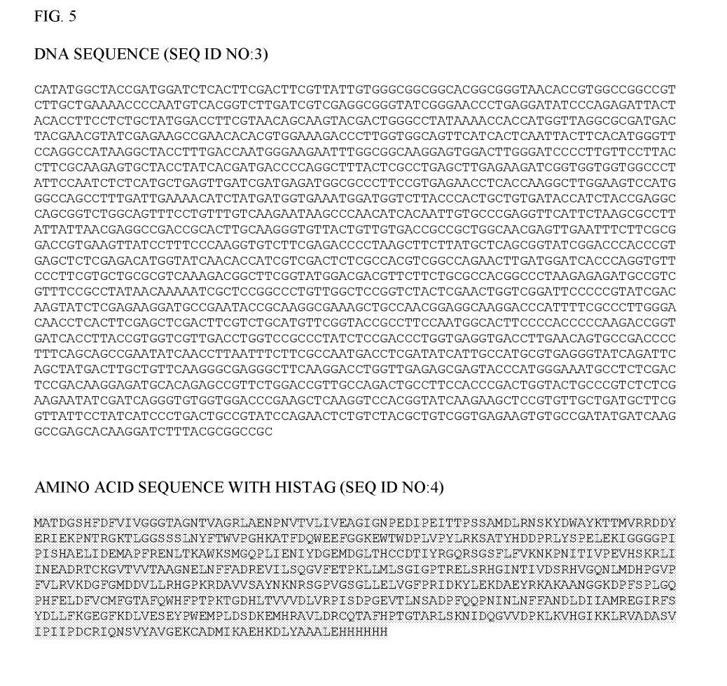

The present invention is not limited to the use of any particular formate oxidase enzyme. Indeed, the use of a variety of formate oxidase enzymes is considered. The formate oxidase enzymes may be isolated from natural sources such as yeast or other fungi or produced recombinantly. Suitable formate oxidases include, but are not limited to those, from Schwanniomyces vannjiae (also known as Debaryomyces vanrijae; SEQ ID NO:1 (DNA sequence), SEQ ID NO:2 (protein sequence), see FIG. 2), Aspergillus oryzae, Genbank ID XM_001727326.1 (SEQ ID NO:3 (DNA sequence), SEQ ID NO:4 (protein sequence), see FIG. 5), as well as formate oxidases from Aspergillus nomius and Paecillomyces variotii. In some embodiments, the formate oxidase is described in Maeda et al. (Acta Cryst. (2010). F66, 1064-1066) and Maeda et al., (Acta Cryst. (2010). F66, 1064-1066) and Kondo (FEMS Microbiology Letters 214 (2002) 137-142); each of which is herein incorporated by reference in its entirety. In some embodiments, the formate oxidase is isolated from the fermentation broth of the organism, while in other embodiments, the enzyme is produced recombinantly in E. coli or the yeast Saccharomyces cerevisiae into which a suitable vector allowing expression of a formate oxidase (e.g., a vector expressing the nucleic acid SEQ ID NO:1 or 3) has been introduced. Where recombinant FOX (rFOX) is produced, the rFOX may be wild-type or variant rFOX. Accordingly, in some embodiments, the FOX utilized in the present invention has at least 90%, 95%, 97%, 98%, 99% or 100% identity with the FOX amino acid sequences SEQ ID NO:2 or SEQ ID NO:4 and has formate oxidase activity. In some embodiments, the formate oxidase enzyme utilized in the present invention does not require the cofactor NAD to catalyze the oxidation for formate to carbon dioxide and hydrogen peroxide. In this way, the formate oxidases utilized in the present invention are distinguished from formate dehydrogenases which require the cofactor NAD for activity. In some embodiments, whether the enzyme is isolated from the native organisms or produced recombinantly, stabilizers such as EPPS buffer pH 8.4 and serum albumin (BSA) are used to protect the isolated and purified FOX enzyme from degrading.

In some embodiments, the presence of formate in a biological sample is detected by utilizing a peroxidase enzyme to catalyze a reaction between the hydrogen peroxide produced by the oxidation of formate by formate oxidase and a colorimetric substrate. Suitable peroxidase enzymes include, but are not limited to, horse radish peroxidase (HRP), soybean peroxidase and other peroxidases known in the art. Suitable chromogenic substrates, also referred to as indicator dye precursors, are known in the in the art and include, but are not limited to, ABTS (2,2′-Azinobis [3-ethylbenzothiazoline-6-sulfonic acid]-diammonium salt), OPD (o-phenylenediamine dihydrochloride), TMB (3,3′,5,5′-tetramethylbenzidine), 4-CN (4-Chloro-1-naphthol), DAB (3,3′-diaminobenzidine), and TMB (3,3′,5,5′-Tetramethylbenzidine). The peroxidase enzyme catalyzes a reaction in the presence of the chromogenic substrate and hydrogen peroxide to produce a detectable colored substrate that can be detected visually or by a photospectometry.

Accordingly, in some embodiments, the present invention provides an assay system suitable for detecting the presence of formate in a biological sample. In some embodiments, the assay system comprises formate oxidase enzyme, a peroxidase enzyme and an indicator dye precursor. In some embodiments, the formate oxidase enzyme, peroxidase enzyme, and indicator dye precursor are provided in separate containers in a kit format. In other embodiments, the formate oxidase enzyme, peroxidase enzyme, and indicator dye precursor are provided in test strip, preferably a dry test strip. In some embodiments, the dry test strips of the present invention remain stable at room temperature for a period of at least 1, 2, 3, 6, 12 or 24 months. In some preferred embodiments, the test strips are stable at least 1, 2, 3, 6, 12 or 24 months without refrigeration.

Accordingly, in some embodiments, a test strip or other dry chemistry system where the biological fluid flows onto the dry reagents is utilized (See e.g., U.S. Pat. Nos. 4,774,192 and 4,877,580; each of which is herein incorporated by reference in its entirety). In some embodiments, the dry test strip has a moisture content of less than 5, 4, 3, 2, 1, 0.5 or 0.1%. In some embodiments, the test strips are configured for flow or capillary assays (e.g., alone or in kit or systems).

For example, in some embodiments, test strips are generated using the methods described in the experimental section. The order of absorption of the constituents of the dry chemistry reagent system into the substrate utilized for the test strip is generally dictated by considerations involving chemical compatibly and/or other factors relating to solubility in a common solvent.

In some embodiments, the test strip of the present invention comprises a porous substrate such as a membrane. The porous substrate is preferably impregnated with dry chemical reagents (e.g., the formate oxidase enzyme, peroxidase enzyme and indicator dye precursor), preferably in a defined reaction zone, that allow detection of an analyte of interest. In some embodiments, the porous substrate in encased in a housing comprising at least one viewing window. In some embodiments, the porous substrate slides within the housing so that it can be viewed through the viewing window and a portion of the substrate extends beyond the housing so that is may be grasped by the user and slid within the housing and/or removed from the housing. In operation of the device, a fluid sample (such as a bodily fluid sample) is placed in contact with the porous substrate. In some embodiments, the device also includes a sample application area (or reservoir) to receive and temporarily retain a fluid sample of a desired volume. In some embodiments, the sample application area facilitates application of a sample to the porous substrate, preferably at sample receptive surface of the porous substrate and adjacent to the reaction zone containing the dry chemistry reagents. The fluid components of the sample pass through the substrate matrix when applied to the porous substrate. In this process, an analyte in the sample (e.g., formate) can specifically interact with the reagents (e.g., dry chemical reagents deposited using the methods described herein), participate in a chemical reaction, and generate a detectable signal. Optional wash steps can be added at any time in the process, for instance, following application of the sample.

In preferred embodiments, the sample receptive surface is essentially impermeable to cells and particulate matter, but allows diffusion of the analyte into the porous substrate so that the analyte may come into contact with the dry chemistry reagents. In some embodiments, the sample receptive surface allows separation of plasma containing the analyte from blood cells and other particulate matter in the blood sample. In some embodiments, the sample is applied to the sample receptive surface of the porous substrate, allowing for adsorption of the fluid fraction of the sample into the matrix of the porous substrate and detection of an indicator molecule (e.g., the indicator dye formed from the indicator dye precursor). In some embodiments, the indicator molecule provides for colorimetric quantitation (e.g., semi-quantitative or quantitative measurement) of the amount of the analyte of interest (e.g., formate) in the sample. In some embodiments, the interaction of the analyte of interest with the reagents in the reaction zone produces a characteristic set of color values that correlate with the presence of specific assay values for a particular analyte for visual comparison and quantitative assessment. In some embodiments, the assay devices further comprise a color comparator including a plurality of different color fields arranged in an ordered, preferably linear, succession, the color intensity of each field connoting a particular assay value for the analyte. In some embodiments, the color comparator is arranged on the housing so that the porous membrane may be moved in relation to the color comparator to match the color of the reaction zone to the corresponding color on the color comparator to connote a particular assay value for the analyte. In some embodiments, the color comparator is provided separately (e.g., on a separate strip) and the particular assay value for the analyte is obtained by comparing the color comparator to the reaction zone on the porous substrate. In some embodiments, where the porous membrane comprises a sample receptive surface, the device may be preferably inverted so that the color is read from the side opposite of the sample receptive surface. In some embodiments, the porous substrate or the porous substrate within the housing can also be inserted into a reflectance meter, a photometer or a fluorometer; and, the reporter molecule measured and compared with a standard curve for the analyte of interest. The instrument will then report a quantitative value based upon its observation and comparison with a standard.

In some embodiments, the porous substrate is conditioned by treatment with a first solution containing protein, glucose, dextrin or dextrans, starch, polyethylene glycol (PEG), polyvinyl pyrrolidone (PVP), or an equivalent. The purpose of such conditioning is two-fold: (a) to effectively reduce the void space within the matrix of the substrate and, (b) to assist or promote the absorption of the fluid fraction of the biological sample. In some embodiments, the conditioning agent is combined with one or more of the interactive materials of the reagent system and concurrently absorbed into the substrate. Where the conditioning agent is combined with the interactive materials of the reagent composition, its absorption by the substrate will necessarily be preceded by absorption of the indicator molecule.

Where such conditioning of the porous substrate is effected independent of the interactive materials of the reagent system, the substrate is dried under controlled conditions, and then contacted with one or more solutions containing assay components, for example, enzymes, substrates, and indicator (or the chemical precursor of the indicator molecule) dissolved in a suitable buffer.

In some embodiments, the solution also contains a “flow control agent”. This agent modulates the rate of spreading/distribution of the fluid fraction of this sample throughout the matrix of the substrate. It is, thus, effective in the prevention of the chromatographic separation of the reagents within the membrane matrix upon the addition of the fluid sample. Following addition of this third solution, the substrate is air dried for removal of excess fluid, lyophilized and shielded from light.

Once the reagent delivery system has been prepared, the resultant substrate impregnated with dry chemistry reagents is utilized in any one of several test strip configurations specific for the analysis of whole blood or other samples.

Experiments conducted during the course of development of embodiments of the present disclosure screened a variety of color indicators, buffers for dissolving assay reagents, surfactants, and additional agents to improve stability of assay components. While not limiting the present disclosure to particular components, in some embodiments for detection of formate, the color indicator TMB (3,3′,5,5′-tetramethylbenzidine) is used. Other relevant indicators include, but are not limited to, ABTS (2,2′-Azinobis [3-ethylbenzothiazoline-6-sulfonic acid]-diammonium salt); OPD (o-phenylenediamine dihydrochloride); 4-chloronaphthol; o-dianisidine; guaiacol (2-methoxyphenol); 4-(N-Methylhydrazino)-7-nitro-2,1,3-benzooxadiazole (MNBDH); 4-Aminoantipyrine (4AAP), or 2,6-dichloroquinone-4-chloroimide (Gibbs' reagent) for reflectance photometry and for example resazurin for fluorescence. In some embodiments, HEPES buffer (pH 8), trehalose and dextran, BioTerge surfactant, are utilized to optimize performance.

The particular materials used in a particular assay strip device are selected to optimize, for example, the desired detection limit and concentration range for the analysis, and hence the sample volume needed, and stability and compatibility with the reagents. In some embodiments, there is a sample pad which receives the sample and retains particulates, in particular red blood cells, from the sample to limit background readings. In some embodiments, the sample pad is cellulose. Sample pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%).

The test strips of embodiments of the present disclosure are not limited to use of a particular substrate. The substrates physical characteristics (tensile strength, thickness, etc.) are of course to be consistent with test strip manufacture; that is, it should have sufficient dimensional stability and integrity to permit sequential absorption and drying of the conditioning agent, the reagent cocktail and/or indicator without loss of its physical strength. The physical attributes of the substrate should also preferably provide sufficient durability and flexibility to adapt in automated processes for continuous manufacturing of test strips. The physical characteristics of the substrate should, in addition, be otherwise consistent with the absorption and retention of aqueous fluids in the contemplated environment of use.

The substrate is preferably relatively chemically inert; that is, essentially unreactive toward both the constituents of the chemistry reagent system and toward the constituents of a sample which is to be reacted with the reagent system within the substrate. It is, however, to be anticipated that certain of the inherent qualities of the substrate surface and/or its matrix may exhibit some affinity for a constituent of the reagent system and/or a constituent of the fluid sample. This natural attraction can, in certain instances, be used to advantage to immobilize a constituent of the reagent cocktail and/or sample on or within a portion of the substrate and thereby effect a type of separation or anisotropic distribution of the constituents of the cocktail/sample.

The substrate's optical properties should also enable effective observation/monitoring of the reaction manifesting indicator species. This requirement would, thus, contemplate that the substrate provide a background of sufficient contrast to permit observation of the indicator species at relatively low concentrations. Where the indicator is a fluorophore, the background fluorescence of the membrane should be minimal or be essentially non-fluorescent at the monitored wavelength of interest.

Where the inherent characteristics of the substrate are not conducive to effective monitoring of an indicator, it may be desirable to introduce a pigment into the dry chemistry reagent system. For example, certain of the membranes which may be potentially suitable for use in this invention can be colored or transparent. The introduction of pigment into the chemistry reagent system provides a suitable background against which to measure the indicator species.

In some preferred embodiments, the substrate utilized the test strips of the present invention is nitrocellulose, nylon, or mixed polymer membrane CQ (IPOC). Further examples of useful substrates include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; and mixtures or copolymers of the above classes, such as graft copolymers obtained by initializing polymerization of synthetic polymers on a pre-existing natural polymer.

It is contemplated that porous substrates described hereinabove are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm.

The surface of a solid support may be activated by chemical processes that cause covalent linkage of an agent (e.g., an assay reagent) to the support. However, any other suitable method may be used for immobilizing an agent to a solid support including, without limitation, ionic interactions, hydrophobic interactions, and the like. The particular forces that result in immobilization of an agent on a solid phase are not important for the methods and devices described herein.

Except as otherwise physically constrained, a substrate may be used in any suitable shapes, such as films, sheets, strips, or plates, or it may be coated onto or bonded or laminated to appropriate inert carriers, such as paper, glass, plastic films, or fabrics.

In some embodiments, assay strip devices of the present invention include a strip of absorbent or porous material (such as a microporous membrane), which, in some instances, can be made of different substances each joined to the other in zones, which may be abutted and/or overlapped. In some examples, the absorbent strip can be fixed on a supporting non-interactive material (such as nonwoven polyester), for example, to provide increased rigidity to the strip.

In some embodiments, a fluid sample (or a sample suspended in a fluid) is introduced to the strip at the sample receptive surface, for instance by dipping or spotting. A sample is collected or obtained using methods well known to those skilled in the art. The sample containing the analyte to be detected may be obtained from any biological source. Examples of biological sources include whole blood, blood serum, blood plasma, urine, spinal fluid, saliva, fermentation fluid, lymph fluid, tissue culture fluid and ascites fluid of a human or animal. The sample may be diluted, purified, concentrated, filtered, dissolved, suspended or otherwise manipulated prior to the assay to optimize the results. The fluid migrates distally from the application point through the functional regions of the strip. The final distribution of the fluid in the individual functional regions depends on the adsorptive capacity and the dimensions of the materials used.

Other useful assay device formats which may be adapted for use in the present invention are described in, e.g., U.S. Pat. No. 4,770,853; PCT Publication No. WO 88/08534 and European Patent No. EP-A 0 299 428, and U.S. Pat. Nos. 5,229,073; 5,591,645; 4,168,146; 4,366,241; 4,855,240; 4,861,711; 4,703,017; 5,451,504; 5,451,507; 5,798,273; 6,001,658; and 5,120,643; European Patent No. 0296724; WO 97/06439; and WO 98/36278, all of which are incorporated herein by reference.

In some embodiments, the present invention provides a kit comprising components useful, necessary, or sufficient for measuring toxins or metabolites there of (e.g., formic acid/formate) in a biological sample (e.g., blood, plasma, serum, or urine). In some embodiments, kits comprise, consist essentially of, or consist of, a oxidase enzyme (e.g., formate oxidase, a peroxidase, an indicator dye precursor (e.g., TMB), positive control, and directions for use. In some embodiments, the oxidase, the peroxidase and the dye precursor and any additional components are embedded on a test strip. In some embodiments, kits comprise reagents for identifying multiple analytes (e.g., ethanol in addition to formate) in a biological sample (e.g., multiple test strips, each of which is specific for a different analyte or a single strip that detects multiple analytes).

In some embodiments, kits are generally portable and provide a simple, rapid, and/or cost-effective way to determine the presence or absence of analytes without the need for laboratory facilities, such as in a point-of-care facility.

In some embodiments, the kits of the present invention include one or more assay devices and optionally a reader or other detection device, as disclosed herein and a carrier means, such as a box, a bag, a satchel, plastic carton (such as molded plastic or other clear packaging), wrapper (such as, a sealed or sealable plastic, paper, or metallic wrapper), or other container. In some examples, kit components will be enclosed in a single packaging unit, such as a box or other container, which packaging unit may have compartments into which one or more components of the kit can be placed. In other examples, a kit includes one or more containers, for instance vials, tubes, and the like that can retain, for example, one or more biological samples to be tested, positive and/or negative control samples or solutions, diluents (such as, phosphate buffers, or saline buffers), detector reagents, and/or wash solutions (such as, buffers, saline buffer, or distilled water).

Other kit embodiments include syringes, finger-prick devices, alcohol swabs, gauze squares, cotton balls, bandages, latex gloves, incubation trays with variable numbers of troughs, adhesive plate sealers, data reporting sheets, which may be useful for handling, collecting and/or processing a biological sample. Kits may also optionally contain implements useful for introducing samples into a sample chamber of an assay device, including, for example, droppers, Dispo-pipettes, capillary tubes, rubber bulbs (e.g., for capillary tubes), and the like. Still other kit embodiments may include disposal means for discarding a used assay device and/or other items used with the device (such as patient samples, etc.). Such disposal means can include, without limitation, containers that are capable of containing leakage from discarded materials, such as plastic, metal or other impermeable bags, boxes or containers.

In some embodiments, a kit of the present invention will include instructions for the use of an assay device. The instructions may provide direction on how to apply sample to the test device, the amount of time necessary or advisable to wait for results to develop, and details on how to read and interpret the results of the test. Such instructions may also include standards, such as standard tables, graphs, or pictures for comparison of the results of a test. These standards may optionally include the information necessary to quantify analyte using the test device, such as a standard curve relating intensity of signal or number of signal lines to an amount of analyte therefore present in the sample.

In some embodiments, the present disclosure provides systems comprising the assay devices described herein; and a detection device. In some embodiments, currently available blood glucose or blood cholesterol measuring devices are utilized to detect levels of toxins or metabolites thereof (e.g., formic acid levels or the presence or absence of formic acid or formate levels (e.g., using the chemistry described herein)). For example, in some embodiments, commercially available blood glucose meters or cholesterol meters from Health Chem, FL with identical or modified calibration of the instrument. In the electrochemical embodiments, existing commercial instruments from Lifescan, Bayer Healthcare, Arkray, and others can be used.

Such meters utilize a test strip (e.g., those described herein). Blood is applied to the test strip. The test strip is inserted into the meter, which then measures the production of hydrogen peroxide by measuring the color intensity of the test field (e.g., spectrophotometrically). In such embodiments, the glucose oxidase or cholesterol oxidase is replaced with formate oxidase. The chemistry described above is then utilized to measure formic acid/formate in blood or urine.

The present invention is not limited to the use of blood glucose meters or cholesterol meters for detection. In some embodiments, the chemistry described herein is applied in capillary microfluidic platforms (See e.g., Chem. Soc. Rev., 2010, 39, 1153-1182; herein incorporated by reference in its entirety), paper-based devices (See e.g., Anal. Chem. 2009, 81, 8447-8452; herein incorporated by reference in its entirety), laboratory test strip readers, or filter paper.

### II. Methods

In some embodiments, the devices, kits, systems and methods described herein find use in monitoring methanol outbreaks in the field. In some embodiments, systems, kits, and methods find use in the developing world where the ability to rapidly and inexpensively detect methanol poisoning in the field is particularly useful. The systems and methods described herein are able to provide a definitive diagnosis of methanol poisoning in two hours or less, one hour or less, 30 minutes or less, 15 minutes or less, or 5 minutes or less or 3 minutes or less, using a drop of blood without relying on laboratory equipment.

The symptoms of methanol poisoning can be difficult to distinguish. In addition, some incidents of methanol poisoning are the result of ethanol that is contaminated with methanol. It is important to be able to rapidly distinguish between acidosis, ethanol intoxication and methanol poisoning in order to administer appropriate treatment. Accordingly, in some embodiments, the systems and methods described herein find use in distinguishing between exposure to methanol and ethanol or metabolic acidosis of unknown or other origin in a subject. Test strips for detection of methanol (e.g., test strips for detection of formic acid/formate) are parts of a diagnostic system to rapidly provide a firm diagnosis of methanol poisoning.

In some embodiments, the systems and methods described herein are used to monitor treatment for methanol poisoning. In some embodiments, methanol poisoning is treated by administration of ethanol or fomepizole. During treatment with ethanol, it is important to closely monitor blood levels of formate to ensure that the treatment is effective. In this embodiment it is mandatory that ethanol in therapeutic concentrations does not interfere with the accurate quantification of formate.

### EXPERIMENTAL

The following examples are provided in order to demonstrate and further illustrate certain preferred embodiments and aspects of the present disclosure and are not to be construed as limiting the scope thereof.

### EXAMPLE 1

This example describes the determination of appropriate dilutions of formate oxidase for use in formate assays and for detection of activity via color change. Three different lots of formate oxidase were tested, lots 0906151, 0906152, and 0906153.

Reagents. All Chemicals are a defined grade (ACS Reagent, U.S.P, N.F, etc). The Horseradish Peroxidase (HRP) Enzyme is an RZ-3 material with confirmed activity. The formate oxidase (FOX) was supplied from GenScript, Piscataway, N.J.; the sequences for the formate oxidase are shown in FIG. 2. Sodium formate was supplied in 5% BSA/saline. DI water is an abbreviation of deionized water.

TABLE 1
Formate oxidase cocktail (Cocktail #15) preparation,
for 50 ml Cocktail Solution

| | | | Quantity | |
|---|---|---|---|---|
| Material | Quantity | | added | |
| 3,3′,5,5′-Tetramethylbenzidine (TMB) | 0.5 | g/L | 0.025 | g |
| Dimethyl Sulfoxide | As needed | | ca 1 | ml |
| Polyvinyl Pyrrolidone 5% | 800 | mI/L | 40 | ml |
| (PVP-40,000 M.V.) | | | | |
| Phosphate Buffer pH 6.5 | 27.2 | g/L | 1.36 | g |
| Sodium Cholate | 1 | g/L | 50 | mg |
| Bovine Serum Albumin (BSA) | 2.0 | g/L | 10 | mg |
| Horseradish Peroxidase Enzyme | 100.000 | U/L | 20.75 | mg |

The enzyme base solution was prepared by weighing out the above materials, using the quantity given in the table 1, in the following order, and letting each component dissolve before adding the next component: Polyvinyl Pyrrolidone 5% (PVP-40,000 M.V.), Phosphate Buffer pH 6.5, Sodium Cholate Hydrate. For the TMB/DMSO solution, the 3,3′,5,5′-Tetramethyl-benzidine was dissolved in as little DMSO as possible in separate container. The TMB/DMSO solution was then added to the enzyme base solution. The pH of the solution was adjusted to 4.6 ±0.1 using 1M Boric Acid. The Bovine Serum Albumin (BSA) was then weighed out, added to the solution, and mixed to dissolve. The required quantity of horseradish peroxidase enzyme was then calculated and added and mixed to dissolve. The solution as then diluted to the desired volume with deionized water, mixed, and filtered or centrifuged as needed.

Working and control solution preparation. To one tube of lyophilized formate oxidase 2, 1 ml of deionized water was added. The tubes were not shook but allowed to sit for 5 minutes. After 5 minutes, the tubes were vortex then allowed to sit for 5 minutes. The following working solutions where then prepared:

1. Lot 0906151 (5.5 mg/ml protein)—to 1 ml of Cocktail # 15 added 50 ul FOX

2. Lot 0906152 (1.2 mg/ml protein)—to 1 ml of Cocktail # 15 added 145 ul FOX

3. Lot 0906153 (0.7 mg/ml protein)—to 1 ml of Cocktail # 15 added 375 ul FOX

4. Lot 0906151 (5.5 mg/ml protein)—to 1 ml of Cocktail # 15 added 25 ul FOX

5. Lot 0906152 (1.2 mg/ml protein)—to 1 ml of Cocktail # 15 added 12.5 ul FOX

6. Lot 0906153 (0.7 mg/ml protein)—to 1 ml of Cocktail # 15 added 5.0 ul FOX

7. Lot 0906151 (5.5 mg/ml protein)—to 1 ml of Cocktail # 15 added 1.0 ul FOX

8. Lot 0906151 (0.7 mg/ml protein)—to 1 ml of Cocktail # 15 added 2.0 ul FOX

Control solutions were prepared as follows:

1. 5.5 mg/ml protein—to 1 ml of Cocktail # 15 added 50 ul DI water

2. 1.2 mg/ml protein—to 1 ml of Cocktail # 15 added 145 ul DI water

3. 0.7 mg/ml protein—to 1 ml of Cocktail # 15 added 375 ul DI water

These reagents were then used to assay various amounts the formate as summarized in the following tables.

TABLE 2
Formate oxidase lot 0905153 (0.7 mg/ml),
Formate oxidase amount 375 ul/ml

| Sodium | | | | |
|---|---|---|---|---|
| Formate | Amount | | | |
| Std | of Std | Color Change Observed | | |
| (mM) | (ul) | 1 min | 5 min | >5 min |
| 40 | 0.92 | blue | dark blue (hint of brown) | brown |
| 20 | 0.92 | blue | dark blue (hint of brown) | brown |
| 10 | 0.92 | blue | dark blue (hint of brown) | brown |
| 5 | 0.92 | blue | dark blue (hint of brown) | brown |
| 2 | 0.92 | light blue | blue | blue |
| 1 | 0.92 | light blue | blue | blue |
| 0.5 | 0.92 | light blue | blue | blue |
| 0.3 | 0.92 | light blue | blue | blue |

TABLE 3
Formate oxidase lot 0905152 (1.2 mg/ml),
Formate oxidase amount 375 ul/ml

| Sodium | | | | |
|---|---|---|---|---|
| Formate | Amount | | | |
| Std | of Std | Color Change Observed | | |
| (mM) | (ul) | 1 min | 5 min | >5 min |
| 40 | 0.92 | blue | dark blue (hint of brown) | brown |
| 20 | 0.92 | blue | dark blue (hint of brown) | brown |
| 10 | 0.92 | blue | dark blue (hint of brown) | brown |
| 5 | 0.92 | blue | dark blue (hint of brown) | brown |
| 2 | 0.92 | light blue | dark blue | dark blue |
| 1 | 0.92 | light blue | dark blue | dark blue |
| 0.5 | 0.92 | light blue | light blue | light blue |
| 0.3 | 0.92 | light blue | light blue | light blue |

TABLE 4
Formate oxidase lot 0905151 (5.5 mg/ml),
Formate oxidase amount 375 ul/ml

| Sodium | | | | |
|---|---|---|---|---|
| Formate | Amount | | | |
| Std | of Std | Color Change Observed | | |
| (mM) | (ul) | 1 min | 5 min | >5 min |
| 40 | 0.92 | dark blue | dark blue (hint of brown) | brown |
| 20 | 0.92 | dark blue | dark blue (hint of brown) | brown |
| 10 | 0.92 | dark blue | dark blue (hint of brown) | brown |
| 5 | 0.92 | dark blue | dark blue (hint of brown) | brown |
| 2 | 0.92 | dark blue | dark blue (hint of brown) | blue brown |
| 1 | 0.92 | light blue | dark blue | dark blue |
| 0.5 | 0.92 | light blue | dark blue | dark blue |
| 0.3 | 0.92 | light blue | dark blue | dark blue |

TABLE 5
Formate oxidase lot 0905151 (5.5 mg/ml),
Formate oxidase amount 375 ul/ml

| Sodium | | | | |
|---|---|---|---|---|
| Formate | Amount | | | |
| Std | of Std | Color Change Observed | | |
| (mM) | (ul) | 1 min | 5 min | >5 min |
| 40 | 0.92 | dark blue | dark blue (hint of brown) | brown |
| 20 | 0.92 | dark blue | dark blue (hint of brown) | brown |
| 10 | 0.92 | dark blue | dark blue (hint of brown) | brown |
| 5 | 0.92 | dark blue | dark blue (hint of brown) | brown |
| 2 | 0.92 | dark blue | dark blue (hint of brown) | blue brown |
| 1 | 0.92 | light blue | dark blue | dark blue |
| 0.5 | 0.92 | light blue | dark blue | dark blue |
| 0.3 | 0.92 | light blue | dark blue | dark blue |

TABLE 6
Formate oxidase lot 0905151 (1.2 mg/ml),
Formate oxidase amount 5 ul/ml

| Sodium | | | | |
|---|---|---|---|---|
| Formate | Amount | | | |
| Std | of Std | Color Change Observed | | |
| (mM) | (ul) | 1 min | 5 min | >5 min |
| 40 | 1.0 | dark blue | brown | brown |
| 20 | 1.0 | dark blue | brown | brown |
| 10 | 1.0 | dark blue | brown | brown |
| 5 | 1.0 | dark blue | brown | brown |
| 2 | 1.0 | dark blue | blue brown | blue brown |
| 1 | 1.0 | light blue | dark blue | dark blue |
| 0.5 | 1.0 | light blue | dark blue | dark blue |
| 0.3 | 1.0 | light blue | dark blue | dark blue |

TABLE 7
Formate oxidase lot 0905151 (5.5 mg/ml),
Formate oxidase amount 1 ul/ml

| Sodium | | | | |
|---|---|---|---|---|
| Formate | Amount | | | |
| Std | of Std | Color Change Observed | | |
| (mM) | (ul) | 1 min | 5 min | >5 min |
| 40 | 1.0 | dark blue | orange | orange |
| 20 | 1.0 | blue | dark blue | dark blue |
| 10 | 1.0 | blue | dark blue | dark blue |
| 5 | 1.0 | blue | dark blue | dark blue |
| 2 | 1.0 | light blue | light blue | light blue |
| 1 | 1.0 | light blue | light blue | light blue |
| 0.5 | 1.0 | light blue | light blue | light blue |
| 0.3 | 1.0 | light blue | light blue | light blue |

TABLE 8
Formate oxidase lot 0905151 (5.5 mg/ml),
Formate oxidase amount 2 ul/ml

| Sodium | | | | |
|---|---|---|---|---|
| Formate | Amount | | | |
| Std | of Std | Color Change Observed | | |
| (mM) | (ul) | 1 min | 5 min | >5 min |
| 40 | 1.0 | dark blue | orange | orange |
| 20 | 1.0 | blue | orange | orange |
| 10 | 1.0 | blue | dark blue | dark blue |
| 5 | 1.0 | blue | dark blue | dark blue |
| 2 | 1.0 | light blue | blue | blue |
| 1 | 1.0 | light blue | blue | blue |
| 0.5 | 1.0 | light blue | light blue | light blue |
| 0.3 | 1.0 | light blue | light blue | light blue |

TABLE 9
Formate oxidase lot 0905151 (5.5 mg/ml),
Formate oxidase amount 2 ul/ml

| Sodium | | | | |
|---|---|---|---|---|
| Formate | Amount | | | |
| Std | of Std | Color Change Observed | | |
| (mM) | (ul) | 1 min | 5 min | >5 min |
| 40 | 1.0 | dark blue | orange | orange |
| 20 | 1.0 | blue | orange | orange |
| 10 | 1.0 | blue | dark blue | dark blue |
| 5 | 1.0 | blue | dark blue | dark blue |
| 2 | 1.0 | light blue | blue | blue |
| 1 | 1.0 | light blue | blue | blue |
| 0.5 | 1.0 | light blue | light blue | light blue |
| 0.3 | 1.0 | light blue | light blue | light blue |

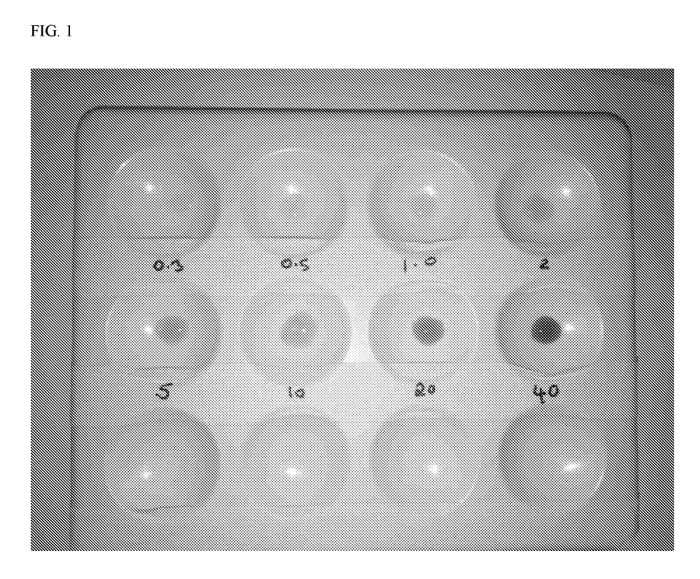

Conclusion. The activity of the three Formate Oxidase 2 lots was verified by means of color change (see FIG. 1). The experiment summarized in Table 9 provides the best color separation and intensity.

### EXAMPLE 2

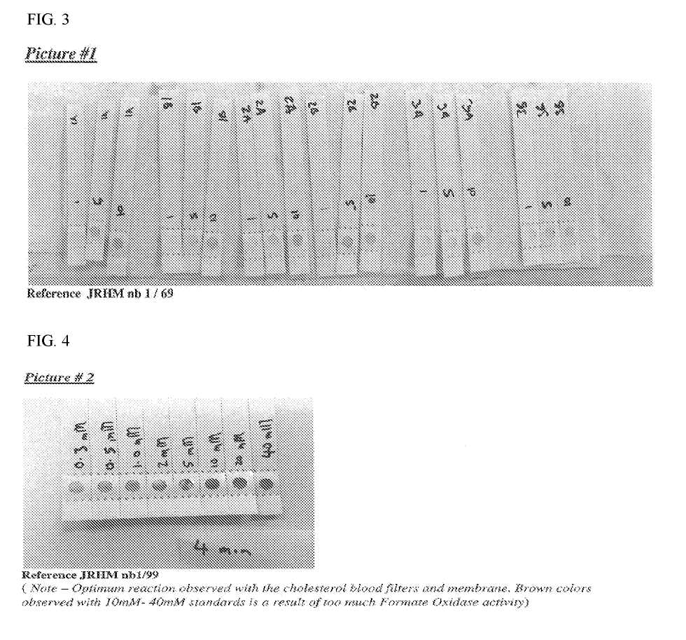

This example describes the production of test strips. The cocktail identified in table 9 above was used to make dry test strips. Formate oxidase was incorporated into the standard detector cocktail #15 including horse radish peroxidase and tetramethylbenzidine and applied to test strips by hand dipping (Pall Corp Polysulphone Biodyne A, six inches wide), dried, and tested toward various concentrations of formate. It can be seen in FIGS. 3 and 4 color development was higher with the higher concentrations of formate and that the cholesterol type of existing analytical system was suitable for the reaction.

### EXAMPLE 3

Recombinant formate oxidase (rFOX) was produced by cloning and expressing Aspergillus oryzae RIB40 glucose-methanol-choline (gmc) oxidoreductase″, gene bank ID XM _001727326.1. Briefly, the restriction sites restriction sites NdeI and NotI were added to the coding sequence (SEQ ID NO: 3, FIG. 5) for the Aspergillus formate oxidase and the construct was subcloned the pET22b vector. E. coli (BL21 (DE3)) was transfected with the plasmid and the E. coli were fermented in terrific broth (TB). Histidine tagged formate oxidase was isolated from the bacterial lysate using Ni-NTA column material from New England Biolabs. . . .

### EXAMPLE 4

The recombinant FOX produced as described in Example 3 was used in assays for formate. Test strips were made with the rFOX as described above in Example 2. The enzyme specificity was tested with buffered solutions and the sensitivity of formate detection using the test strips was examined by spiking blood samples with formate or other test compounds.

The specificity-testing was performed with: D-L-lactate (20 mmol/L), betahydroxy-butyrate (20 mmol/L), glycolate (20 mmol/L), pyroglutamate (20 mmol/L), ascorbic acid (1 mmol/L), ethanol (75 mmol/L), methanol (105 mmol/L), ethylene glycol (20 mmol/L), isopropanol (50 mmol/L), glycerol (3 mmol/L), di-ethylene glycol (10 mmol/L), acetone (55 mmol/L), semicarbacide (1000 mmol/L), glycolic acid, (20 mmol/L), oxalic acid (250 mmol/L), methylene blue (50 umol/L), fomepizole (1 mmol/L), and EDTA (3 mg/ml). There was no influence on the specificity testing on any of the tested substances (i.e. no false positives).

The sensitivity testing was performed with full blood added increasing concentration of formate of 0-1-2-5-10-20 mmol/L. The color reaction was stronger the higher concentration measured, with light blue for the low concentration, more saturated blue for the intermediates, and red for the highest concentrations.

The blinded samples were performed on one ICU nurse and three intensivists, each were given 12 spiked samples of full blood containing various amounts of formate. One of the tested persons was colorblind. The blinded tests indicated that observers could easily distinguish between strongly positives and negative samples.

Although a variety of embodiments have been described in connection with the present disclosure, it should be understood that the claimed invention should not be unduly limited to such specific embodiments. Indeed, various modifications and variations of the described compositions and methods of the invention will be apparent to those of ordinary skill in the art and are intended to be within the scope of the following claims.

1-16. (canceled)

17. A method for detecting methanol poisoning in a subject suspected of being poisoned with methanol, comprising:
a) contacting a biological sample from said subject with a formate oxidase enzyme wherein said formate oxidase acts on formate in said biological sample to produce hydrogen peroxide; and
b) detecting said hydrogen peroxide by producing a colored reagent by reacting said hydrogen peroxide with a peroxidase enzyme and an indicator dye precursor to produce said colored reagent;
wherein the production of the colored reagent is indicative of methanol poisoning in the subject.

18. Method of claim 17, wherein said formate oxidase is selected from the group consisting of an Aspergillus formate oxidase, a Debaromyces formate oxidase, and a Paecillomyces formate oxidase.

19. Method of claim 17, wherein said formate oxidase is a recombinant formate oxidase.

20. Method of claim 17, wherein said formate oxidase exhibits activity upon reconstitution from a dried form on a solid or porous substrate.

21. Method of claim 17, wherein said biological sample is blood, serum, plasma, or urine.

22. Method of claim 17, wherein said indicator dye precursor is selected from TMB and ABTS.

23. Method of claim 17, wherein said peroxidase enzyme is horse radish peroxidase.

24. Method of claim 17, wherein said formate oxidase, said peroxidase and said indicator dye precursor are embedded in a test strip.

25. Method of claim 24, wherein said test strip is selected from the group consisting of nitrocellulose membranes, nylon membranes, and mixed polymer membrane CQ (IPOC).

26. Method of claim 24, wherein said test strip forms a flow through assay.

27. Method of claim 17, wherein said colored reagent is detected photometrically.

28. Method of claim 17, wherein said colored reagent is detected using a blood glucose meter or blood cholesterol meter.

29. Method of claim 17, wherein said colored reagent is detected visually.

30. Method of claim 17, wherein said formate oxidase is used to detect formate in a concentration in said biological sample of from 1 mM to 12 mM and higher.

31. Method of claim 17, wherein a detectable amount of the colored reagent is produced within from about 30 seconds to 10 minutes.

32. Method of claim 17, wherein the amount of colored reagent produced is quantitative for the amount of formate in said biological sample.

33. Method claim 17, further comprising the step of treating said subject for methanol poisoning when formic acid is present in said biological sample.

34. Method of claim 33, wherein said treatment is ethanol or fomepizole.

35. A method for detecting formate in a biological sample from a subject, comprising:
a) contacting a biological sample with formate oxidase such that formate in said biological sample reacts with said formate oxidase to generate hydrogen peroxide; and
b) detecting said hydrogen peroxide.

36. A device comprising a substrate having thereon dried formate oxidase in an amount sufficient to oxidize formate in a biological sample when contacted with said biological sample in the presence of a reporting system to produce a detectable signal corresponding the presence of formate in the sample.

37-56. (canceled)

57. An assay device, comprising:
a porous substrate comprising a) a formate oxidase polypeptide; b) a peroxidase enzyme; and c) an indicator dye precursor.

58-62. (canceled)
